(19) United States
(12) Patent Application Publication
(10) Pub. No.: US 2002/0115931 A1
(43) Pub. Date: Aug. 22, 2002

(54) Localizing intravascular lesions on anatomic images

(76) Inventors: H. William Strauss, Redwood City, CA (US); Leon Kaufman, San Francisco, CA (US); Vartan Ghazarossian, Menlo Park, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/791,499

(22) Filed: Feb. 21, 2001

(51) Int. Cl. A61B 5/05

(52) U.S. Cl. 600/420

(57) ABSTRACT

Methods and systems for localizing intravascular lesion in a body lumen. In exemplary embodiments, the methods comprise providing or acquiring an image of the body lumen and displaying information about the lesion with or on the image. In some configurations the information is obtained with an intravascular catheter.

### CROSS-REFERENCES TO RELATED APPLICATIONS

The present application is related to patent application Ser. No. 09/670,412, filed Sep. 26, 2000, entitled “Methods and Apparatus for Characterizing Lesions in Blood Vessels and Other Body Lumens,” patent application Ser. No. 09/754,103 filed Jan. 3, 2001, entitled “Intravascular Imaging Catheter,” patent application Ser. No. 09/754,074, filed Jan. 3, 2001, entitled “Position Sensitive Imaging Catheter,” and patent application Ser. No. 09/754,822, filed Jan. 3, 2001, entitled “Position Sensitive Imaging Catheter Having Scintillation Detector,” the complete disclosures of which are incorporated herein by reference.

### BACKGROUND OF THE INVENTION

The present invention relates generally to medical devices and methods. More particularly, the present invention relates to devices and methods for displaying information about intravascular lesions over an anatomic image of the body lumen.

Coronary artery disease resulting from the build-up of atherosclerotic plaque in the coronary arteries is a leading cause of death in the United States and worldwide. The plaque build-up causes a narrowing of the artery, commonly referred to as a lesion, which reduces blood flow to the myocardium (heart muscle tissue). Myocardial infarction (better known as a heart attack) can occur when an arterial lesion abruptly closes the vessel, causing complete cessation of blood flow to portions of the myocardium. Even if abrupt closure does not occur, blood flow may decrease resulting in chronically insufficient blood flow which over time can cause significant tissue damage.

Plaques which form in the coronaries and other vessels comprise inflammatory cells, smooth muscles cells, cholesterol, and fatty substances, and these materials are usually trapped between the endothelium of the vessel and the underlying smooth muscle cells. Depending on various factors, including thickness, composition, and size of the deposited materials, the plaques can be characterized as stable or vulnerable. The plaque is normally covered by an endothelial layer. When the endothelial layer is disrupted, the ruptured plaque releases highly thrombogenic constituent materials which are capable of activating the clotting cascade and inducing rapid and substantial coronary thrombosis. Such rupture of a vulnerable plaque and the resulting thrombus formation can cause vulnerable angina chest pain, acute myocardial infarction, sudden coronary death, and stroke. It has recently been proposed that plaque instability, rather than the degree of plaque build-up, should be the primary determining factor for treatment selection.

A variety of approaches for distinguishing stable and unstable plaque in patients have been proposed. Some of the proposals involve detecting a slightly elevated temperature (approximately 2° Fahrenheit) within vulnerable plaque resulting from inflammation. Other techniques involve exposure of the plaque to infrared light. It has also been proposed to introduce radiolabeled materials which have been shown by autoradiography to bind to stable and vulnerable plaque in different ways.

External detection of the radiolabels, however, greatly limits the sensitivity of these techniques and makes it difficult to determine the precise locations of the affected regions. For example, angiography is very effective in locating lumen-intruding lesions in the coronary vasculature, but provides little or no information concerning the nature and distribution of the lesion. To provide better characterization of the lesion(s), a variety of imaging techniques have been developed for providing a more detailed view of the lesion, including intravascular ultrasound (IVUS), angioscopy, laser spectroscopy, computed tomography (CT), magnetic resonance imaging (MRI), and the like. Thus far, none of these technologies has possessed sufficient sensitivity or resolution necessary to reliably characterize and image the distribution of the plaque at the cellular level in the patient. In particular, such techniques provide little information on whether the plaque is stable or vulnerable.

For all of these reasons, it would be desirable to provide improved methods and apparatus for distinguishing between stable and vulnerable plaque within the coronary and other patient vasculature. It would be further desirable if such methods and techniques could provide an accurate image of the azimuthal distribution of the plaque within the body lumen.

### DESCRIPTION OF BACKGROUND ART

U.S. Pat. No. 6,038,468 describes localizing a catheter in a body lumen using acoustic transducers and synthesizing an image of the body lumen from the acoustic transfer functions. U.S. Pat. No. 5,811,814 discusses detecting lesions with a scintillating detector. U.S. Pat. No. 5,054,492 describes an ultrasonic imaging catheter having markers that have a unique appearance under fluoroscopy depending on the rotational orientation of the catheter. U.S. Pat. No. 4,595,014 describes an imaging probe that can obtain a two-dimensional cylindrically mapped image of the distribution of radiation sources around the probe.

### SUMMARY OF THE INVENTION

The present invention provides improved systems and methods for displaying a lesion in a body lumen. In exemplary embodiments, the present invention displays information about the lesion over an image of the body lumen. In particular, the present invention can illustrate the azimuthal and longitudinal distribution of early stage, vulnerable coronary artery plaque over an image of a coronary blood vessel. Typically the information is provided in a substantially spatially correct position on the image of the blood vessel to allow the operator to easily assess the distribution of the lesion within the blood vessel.

The information obtained from the body lumen will typically provide spatially distributed information about the lesion that would not generally be viewable under fluoroscopy. The information can then displayed in a spatially correct orientation on a separately generated image of the body lumen.

The image of the body lumen can be externally or internally generated. Typically however, the anatomic image of the body lumen is obtained with an external, image capture system such as angiography or fluoroscopy. While angiography is one preferred embodiment because of its simplicity, cost effectiveness, speed, and superior frame rate resolution, it is equally possible to obtain the anatomic image of the body lumen using other imaging methods and systems. For example, other image capture systems include nuclear medicine imaging, computed tomography (CT), magnetic resonance imaging (MRI), ultrasound, electron beam computed tomography, or the like can be used.

In exemplary embodiments, the characterization of the lesion distribution is implemented in situ, i.e., within the body lumen being assessed, and can interrogate the body lumen over a relatively long distance to characterize the disseminated lesion in an efficient fashion. The methods and devices can provide real time, highly sensitive detection so that even minor differences between regions of plaque or other lesions can be determined.

For example, an intravascular catheter having detectors can be percutaneously introduced into the body lumen and advanced to the target region to acquire real time information and/or images of the lesion in the body lumen. A contrast medium can be released from the catheter to localize the position of the catheter during transit through the body lumen. The released contrast medium opacifies the body lumen and allows fluoroscopic images of the body lumen to be obtained. Unfortunately, the contrast medium delivered into the body lumen will diffuse over time and the image of the opacified body lumen will be lost. Consequently, the images of the body lumen can be saved in a computer memory to create “ghost images” of the body lumen that can later be recalled to create a background for the data obtained by the detectors. For a more complete discussion of “ghost” imaging of body lumens, see for example, Kaufman L, Kramer D M and Hawryszko C., U.S. Pat. No. 5,155,435 entitled “Method and Apparatus For Performing Interventional Medical Procedures Using MR Imaging Of Interventional Device Superimposed with Ghost Patient Image”; Georgian-Smith D, Goldhaber D M, Haynor D R and Kaufmnan L, “Ghost Imaging for Targeting Breast Masses with MR Imaging,” Academic Radiology 7: 487, 2000; and Kaufmnan L, Goldhaber D M, Kramer D M, Hawryszko C, Georgian-Smith D and Haynor D, “Ghost Imaging in MRI,” MEDICINE MEETS VIRTUAL REALITY 2001”, Westwood J D, Hoffmnan H M, Mogel G T, Stredney D and Robb R A, Eds., IOS Press, Oxford, England, 2001, p.229-235, the complete disclosures of which are incorporated herein by reference.

A coordinate system can be created for the body lumen to allow the data obtained by the catheter to be displayed with the anatomic image. To create the coordinate system for the body lumen, the user can track the position of markers or other fiducials on the catheter and fit a curve in the body lumen that accurately reflects the curvature of the body lumen. The systems of the present invention can use a tracing algorithm that is manual, computer-aided, or the like to create the coordinate system or a manual fitting program that allows the user to mark a few points (typically three or more) along the body lumen. The fitting program will fit straight lines or various order curves (second order curve, third order curve, forth order curve, or the like) between the points marked by the operator. The operator can manually interact with the final curve to fit the curve to the body lumen. If a bi-plane angiography is used, this process can be repeated for both planes, so that the vessel coordinate system can be traced in a three-dimensional space.

Rigid and non rigid movements of the patient can affect the ability of the system to correctly superimpose the position of the catheter onto the stored anatomic image. Consequently, various methods can be used to correct for the rigid and non-rigid movement of the patient. For example, one method comprises placing a plurality of fixed fiducials or markers on the patient or patient platform to create a frame of reference for the anatomic images and the information acquired with the catheter. The anatomic images of the body lumen or images of the catheter may be rescaled, shifted, rotated, or the like so as to match the frames of reference of the images. To correct for nonrigid motion, the position of the plurality of fiducials can be tracked relative to each other. If the position of the fiducials become distorted during imaging, the computer can rescale the image to correct for the distortion of the acquired image so that the two images can be correctly registered.

Depending on the type of information that is desired, the catheters of the present invention can use detectors for measuring different characteristics about the lesion. Many embodiments of the catheters will include an array of position sensitive detectors that can transmit information related to azimuthal and longitudinal distribution of the lesion and standard deviation information related to the obtained data.

It should be appreciated that the information obtained with the detectors is usually not anatomic or structural in nature, but instead is directed more toward obtaining the characteristics and spatial distribution of the lesion. In some exemplary embodiments, the detectors will include radiation detectors that can detect radiation counts of a radiolabeled lesion. Other detectors can obtain information related to the temperature distribution of the body lumen and lesion, x-ray density, distribution of paramagnetic markers, distribution of ferromagnetic or iodinated contrast agents, ultrasound signals, infrared or optical signatures, MRI parameters (e.g., signal, T1, T2, hydrogen density, lipid content, water content, susceptibility, diffusion coefficient), or the like.

Once the detectors have obtained information about the lesion, the coordinate system has been defined, and the images have been registered, the saved anatomic image can be recalled and the information acquired by the catheter can be processed and displayed with the anatomic image. The information obtained with the detectors can be displayed in a variety of ways. For example, the resulting image of the body lumen can include a single image or multiple images that include histogram bars or graphs to indicate the longitudinal and azimuthal distribution of the detected information (e.g. markers, temperature, MRI parameters, etc.), a color map indicating the distribution of the lesion, images having a varying brightness to indicate a distribution of the lesion, a three-dimensional view of the body lumen that can illustrate the distribution of the lesion, standard deviation marks in the form of bars or lines, cross-sections of the body lumen showing the azimuthal distribution of the lesion, or the like.

In one particular aspect, the present invention provides a method of localizing a lesion in a body lumen. The method comprises providing an image of the body lumen. Information is acquired about the lesion with a detecting device, and the information is displayed in a spatially correct distribution relative to the image of the body lumen.

The information can include count activity concentration levels, azimuthal and longitudinal distribution of the lesion in the body lumen, and the like. Such a display provides the physician with a map of the lesion within the body lumen.

In an exemplary configuration, the intravascular lesion detected is vulnerable plaque. The vulnerable plaque can be marked with a radiopharmaceutical or other marker which can localize on the vulnerable plaque such that insertion of a radiation detector into the body lumen can locate the vulnerable plaque. The radiopharmaceutical can be delivered to the vulnerable plaque through localized delivery, systemic delivery, or the like.

In another aspect, the present invention provides a method of characterizing a vulnerable plaque lesion in a body lumen. The method comprises imaging the body lumen to obtain an anatomic image of the body lumen. Detector(s) are placed in the body lumen to obtain information about the plaque to distinguish vulnerable plaque from stable lesions and display the information on the anatomic image.

In a further aspect, the present invention provides a system for displaying an anatomic image of a body lumen. The system comprises an imaging device that can acquire an anatomic image of the body lumen and a detecting device to obtain information of a marked lesion in the body lumen. A computer can be configured to store the anatomic image(s) of the body lumen, receive the information from the detecting device, and display the information on the stored anatomic image.

In still another aspect, the present invention provides a method of displaying information about a lesion in a body lumen. The method comprises providing an anatomic image of the body lumen and acquiring azimuthal distribution information about the lesion. The azimuthal distribution information is displayed with the anatomic image of the body lumen.

In another aspect, the present invention provides a system for localizing lesions in a body lumen. The system comprises a catheter body comprising at least one detector that can obtain information about the lesion. A plurality of markers are positioned on the distal portion of the catheter body that allow a user to track the azimuthal orientation of the distal portion of the catheter body. A computer is coupled to the detector. The computer is configured to superimpose the information obtained by the detector on an anatomic image of the body lumen.

In another aspect, the present invention provides a system for displaying an anatomic image of a body lumen. The system includes an imaging device that can acquire an anatomic image of the body lumen. A detecting device can obtain information of a marked lesion in the body lumen. A computer can be configured to store the anatomic image(s) of the body lumen, receive the information from the detecting device, and display the information on the stored anatomic image.

In yet another aspect, the present invention provides a system for localizing lesions in a body lumen. The system comprises imaging means for obtaining an image of the body lumen, detecting means for characterizing the lesion in the body lumen and means for storing the image of the body lumen and superimposing information about the lesion onto the image of the body lumen.

As will be appreciated by those versed in the art, while the present invention will find particular use in the diagnosis of lesions within blood vessels, the present invention will also be useful in a wide variety of diagnostic and therapeutic procedures. The methodology of plaque detection can be extended to the detection of malignancies following the administration of a metabolic or specific radiolabeled agents (e.g., labeled amino acids, labeled glucose, labeled nucleotides and nucleosides, or the like). Examples of such applications include the differentiation of malignant from benign polyps following virtual colonoscopy and of lung carcinoma from benign anatomy following lung screening by X-ray CT or by MRI.

The above and other features of the present invention may be more fully understood form the following detailed description, taken together with the accompanying drawings, wherein similar reference characters refer to similar elements throughout.

### BRIEF DESCRIPTION OF THE DRAWINGS

FIG. 1 is a simplified schematic of a system incorporating the concepts of the present invention;

FIG. 2A is a simplified data capture device having a single detector;

FIG. 2B is a simplified data capture device having a plurality of detectors;

FIG. 3 is a coronary arteriogram having a contrast medium in the body lumen that allows the body lumen to be visible under fluoroscopy;

FIG. 4 shows the relative separation between fiducials on the catheter;

FIG. 5 shows a radiographic image in which the distances between the fiducials on the catheter has a reduced separation in an imaging plane;

FIG. 6 illustrates three points A, B, C marked along the coordinate line in a body lumen to create a coordinate system for the body lumen;

FIG. 7 illustrates additional points D, E, F to increase the accuracy of the coordinate line;

FIG. 8 illustrates is a simplified anatomic image with the lesion indicated by a color map superimposed onto the anatomic image;

FIG. 9 shows an anatomic image having coordinate line having concentration or count activity on a first side of the line and azimuthal distribution information on a second side of the coordinate line;

FIG. 10 is a display of a cross sectional view of the body lumen with bars for indicating the distribution of the lesion;

FIG. 11 illustrates a display which shows a “narrowing” of the body lumen by providing bars that indicate the presence of plaque in the body lumen;

FIG. 12 illustrates a display having two images in which the first image indicates the level of plaque and the second image indicates azimuthal distribution of the plaque;

FIG. 13 illustrates a display of an image in which brightness intensity of the body lumen indicates a level of plaque in the body lumen, whereby the illustration the relative brightness is indicated by a varying line thickness; and

FIG. 14 shows a display of an image enhanced with standard deviation marks along the anatomic image.

### DESCRIPTION OF THE SPECIFIC EMBODIMENTS

The present invention provides improved methods and apparatus for localizing and displaying lesions in body lumens, and in particular for displaying the distribution of vulnerable plaque in blood vessels. The methods of the present invention rely on acquiring a separate image of at least the target portion of the body lumen and superimposing information obtained about the lesion over the separately generated anatomic image. The information about the lesion can include, azimuthal distribution of the lesion, longitudinal distribution of the lesion in the body lumen, concentration or severity of the lesion, the type of lesion, biological activity occurring in the body lumen, temperature of the lesion, radiation counts, MRI parameters (signal, T1, T2, Hydrogen density, lipid content, water content, susceptibility, diffusion coefficient, and so on), x-ray density, paramagnetic, ferromagnetic or iodinated contrast agents, ultrasound signal, infrared or optical signature, and the like.

In exemplary embodiments, an external imaging method, such as fluoroscopy, angiography, x-ray imaging, nuclear medicine imaging, computed tomography (CT), magnetic resonance imaging (MRI), ultrasound, electron beam computed tomography, or the like are used to obtain an anatomic image of the body lumen.

In most embodiments, the information about the lesion is obtained with an intravascular catheter that has been percutaneously and/or transluminally introduced into the body lumen and advanced to the target region. The information about the lesion can then be transmitted to a computer or other processing device for displaying the information about the lesion over the anatomic image.

In a specific use, the present invention can localize and image vulnerable plaque disposed in vascular body lumens and other blood vessels. Vulnerable plaque can be localized using a variety of methods and devices such as measuring temperature, measuring the cholesterol content of the lesion, measuring other biological content of the inflammatory cells of the lesion, or the like.

In some exemplary embodiments, the vulnerable plaque lesion in the body lumen can first be marked to allow the catheter (or other detection device) to better localize the position of the vulnerable plaque. For example, a labeled marker, such as a radiolabeled marker with a binding agent, can be introduced into the patient's blood vessel in such a way that the marker localizes within the lesion or target region which enables assessment of the type of plaque disposed within the blood vessel. Introduction of the labeled marker can be systemic (e.g., oral ingestion, injection or infusion to the patient's blood circulation, and the like), through local delivery (e.g. by catheter delivery directly to a target region within the blood vessel), or a combination of systemic and local delivery. The marker can also be a fluorescent dye, an iodinated contrast agent, a para- or ferromagnetic agent, and the like.

After introduction of the marker to the patient, the marker can be taken up by the lesion at the target region and the amount of the marker, rate of uptake, distribution of the marker, or other marker characteristics can be analyzed to evaluate the distribution and severity of the lesion. The types of radio tracers and radio labels are more fully described in co-pending U.S. patent application Ser. No. 09/670,412, filed Sep. 26, 2000, and titled “Methods and Apparatus for Characterizing Lesions in Blood Vessels and Other Body Lumens,” licensed to the assignee of the present application, the complete disclosure of which was previously incorporated herein by reference.

While the following discussion focuses on methods of imaging radiolabeled vulnerable plaque with radiation detectors, it should be appreciated by those of ordinary skill in the art that the vulnerable plaque (or other types of lesions) can be characterized without using markers or using other types of detectors and markers. For example, the detectors may be configured to measure temperature, biological activity of the lesion, or the like. Moreover, the vulnerable plaque can be marked with optical fluorescence labels, paramagnetic or ferromagnetic agents, and analyzed using detectors that are configured to track the selected marker.

FIG. 1 illustrates an exemplary imaging system 10 incorporating the present invention. The system 10 will typically include an image capture apparatus 12 that can capture an anatomic image of the body lumen. The image capture apparatus can include systems which generate angiographic images, CT images, MRI images, ultrasound images, nuclear medicine images, electron beam computed tomography images, or the like. The image capture apparatus 12 will typically be coupled to a computer 14 that has a processor and memory for processing and storing the anatomic image(s) of the body lumen. The computer may include input devices 18 such as a keyboard, a voice recognition system, a joystick, a mouse, buttons, foot pedals, or the like. A detecting device 16 such as an intravascular imaging catheter can also be coupled to the computer 14 so as to feed data acquired with the catheter detectors into the computer 14. The computer 14 will be programmed to superimpose or otherwise display the information acquired by the catheter over (or adjacent) the anatomic image to display the information about the lesion in the body lumen on a display 20. In many embodiments the information will be placed over the body lumen in the correct anatomical position.

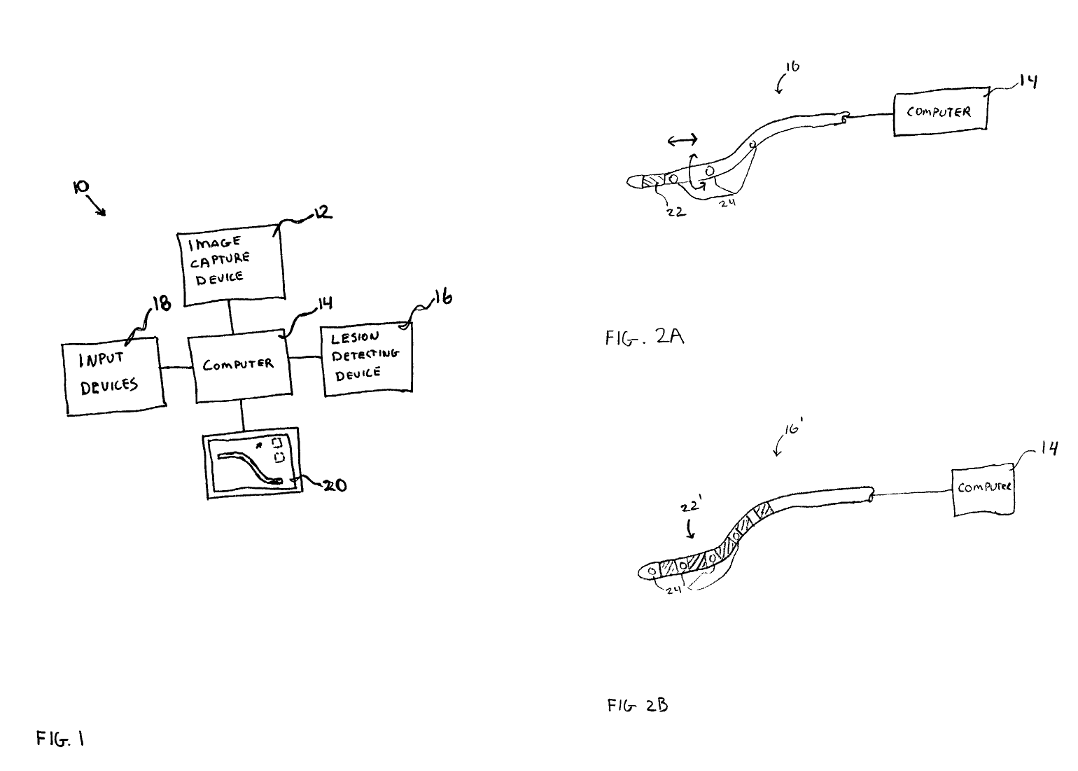

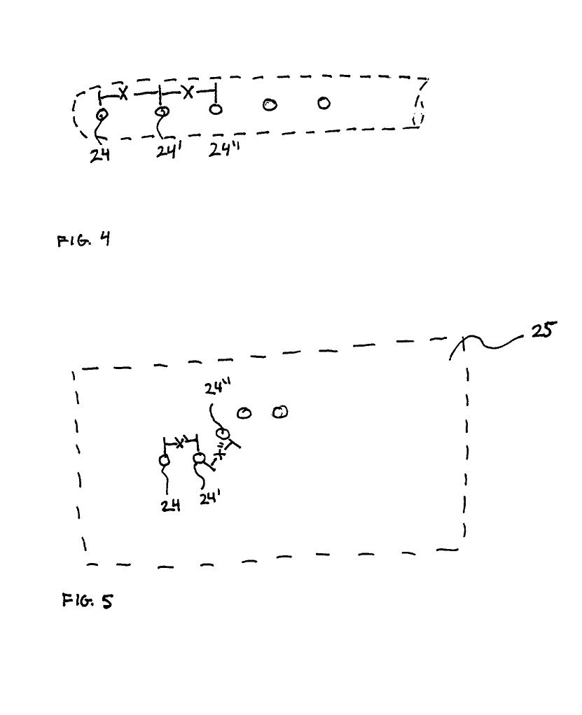

FIGS. 2A and 2B illustrate two exemplary embodiments of an intravascular detecting device 16. The detecting device 16 is typically an intravascular catheter that can be advanced through the body lumen to the target region, typically over a guidewire (not shown). The intravascular catheters 16 include detectors 22 that are coupled to a memory and processor in the computer 14. The information acquired by the detectors 22 can include information regarding the longitudinal and azimuthal distribution of the lesion, concentration and severity of the lesion, the type of lesion, or the like. The detectors 22 can include heat detectors, radiation detectors, optical or infrared detectors, IVUS transducers, MRI coils, ils, pH electrodes, transmembrane potential, or the like. In exemplary embodiments, at least a portion of the catheter 16 absorbs x-rays so that the movement of the catheter can be tracked under radiographic guidance.

As shown in FIG. 2A, the catheter 16 can have a single collimated or uncollimated detector 22 disposed adjacent the distal end of the catheter. The catheter can be advanced at the target region using conventional methods. Once at the target region, the catheter 16 can be pulled or pushed through the vessel (either manually or computer controlled) at a substantially constant longitudinal speed (or alternatively the detector can be pulled within a stationary catheter). The computer 14 can be programmed to track the speed and position of the catheter in the body lumen and display the information onto the anatomic image in an anatomically correct position.

In the embodiments illustrated in FIG. 2B, the catheter 16′ can include a collimated or uncollimated detector array 22′. In such embodiments, the catheter can be positioned at the target region in a longitudinally stationary position within the body lumen during imaging of the count activity. Thus, it may be possible to image the entire lesion without longitudinally moving the catheter 16′. It should be appreciated however, that in other methods the catheter 16′ can be pulled out at a known speed to convert the time of travel into the length of the body lumen.

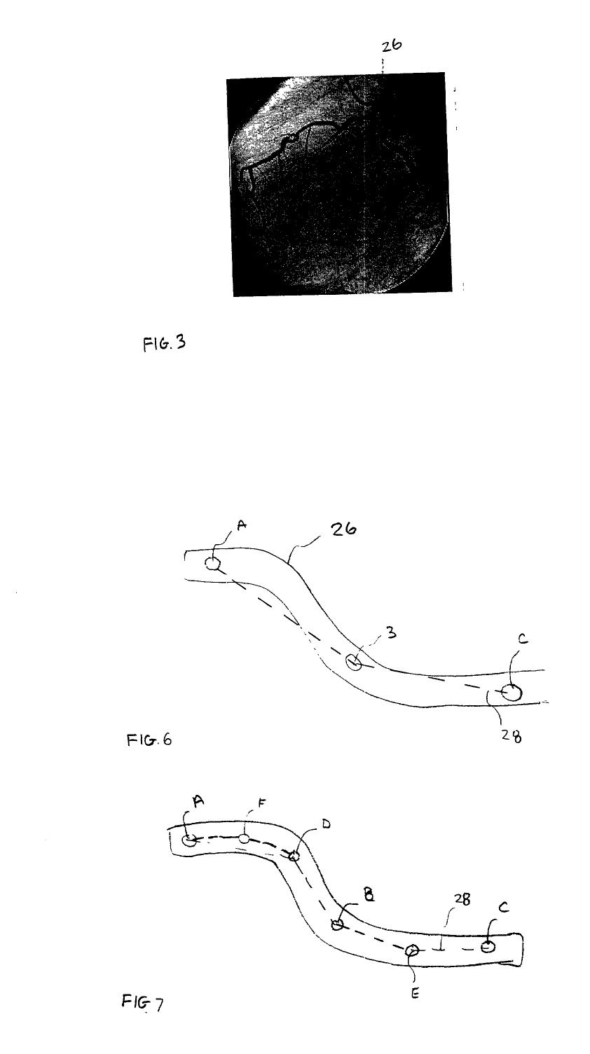

In exemplary methods, a contrast medium is delivered into the body lumen 26 to facilitate the tracking of the catheter during transit to the target region. The contrast medium further assists in the creation of the anatomic image of the body lumen by making the body lumen radiopaque under fluoroscopic imaging (FIG. 3). The images of the radiopaque body lumen will be stored in a memory of the computer 14 for future reference and subsequent combination with ongoing real-time images of the catheter and lesion.

Once the catheter reaches the target portion, the contrast medium will likely have already diffused, and the body lumen will no longer be viewable under fluoroscopic imaging. The images stored by the computer can be searched and/or combined to create a maximum attenuation projection image of the body lumen to create a “ghost image” of the body lumen that can be displayed with the real time images of the catheter.

Because it can be difficult to view the fiducials 24 on the catheter when the body lumen is opacified, to determine the position of the catheter 16 the operator typically must wait for the contrast medium to diffuse before being able to view the radiopaque fiducials. Thereafter, the “ghost image” of the body lumen and the real time image of the catheter can be displayed together. As will be described in more detail below, since breathing and heart motion can displace the saved image of the body lumen and the real-time image of the body lumen matching means, such as scaling software, image rotation and displacement software can be incorporated into the computer 14 to register the saved image of the body lumen and real-time images obtained of the catheter.

During transit of catheter 16 through the body lumen, the radiation detectors may be in a counting mode. Consequently, for catheters with radiation detectors 22, the radiographic based image capture devices 12 can not image body lumen without interfering with the detectors. Thus, in such embodiments, the computer can be programmed to alternate between activating the counting mode in the detectors 22 and imaging the body lumen with image capture device 12. In other embodiments, the “ghost image” can be used to track the position of the catheter, and the detectors can be in the counting mode throughout its transit through the body lumen.

While the catheters of the present invention are able to acquire azimuthal and longitudinal distribution information of the lesion, the catheter merely determines the position and orientation of the lesion relative to the catheter detector(s). Thus, the catheter detectors can first be localized with respect to the body lumen in which it is placed. The absolute azimuthal orientation of the lesion can be obtained by rotating the detector(s) and/or catheter until radiopaque fiducials or markers 24 are viewable in an angiographic image. The catheters will typically include two or more, and preferably between three to five fiducials.

The fiducials are typically composed of magnetic material (for GPS technology) or tantalum, gold, platinum, or other inert heavy materials for x-ray tracking, so that the orientation and position of the catheter can be tracked under radiographic imaging. In most embodiments, the fiducials 24 will have a shape that allows the user to determine the azimuthal orientation of the catheter through the radiographic image. Once the absolute azimuthal orientation is determined the catheter can be maintained in a stationary position to image the target region of the body lumen. Alternatively, if desired the catheter can be moved longitudinally through the target region to obtain information about the lesion. During movement through the target region of the body lumen, the azimuthal orientation of the catheter may change, e.g. the catheter may twist during transit through the body lumen. Consequently, it may be necessary to track the orientation of the radiopaque fiducials or markers 24 that are disposed on the catheter. The fiducials 24 can be tracked manually through visualization of real-time radiographic images of the catheter or through automatic computer tracking of the fiducials.

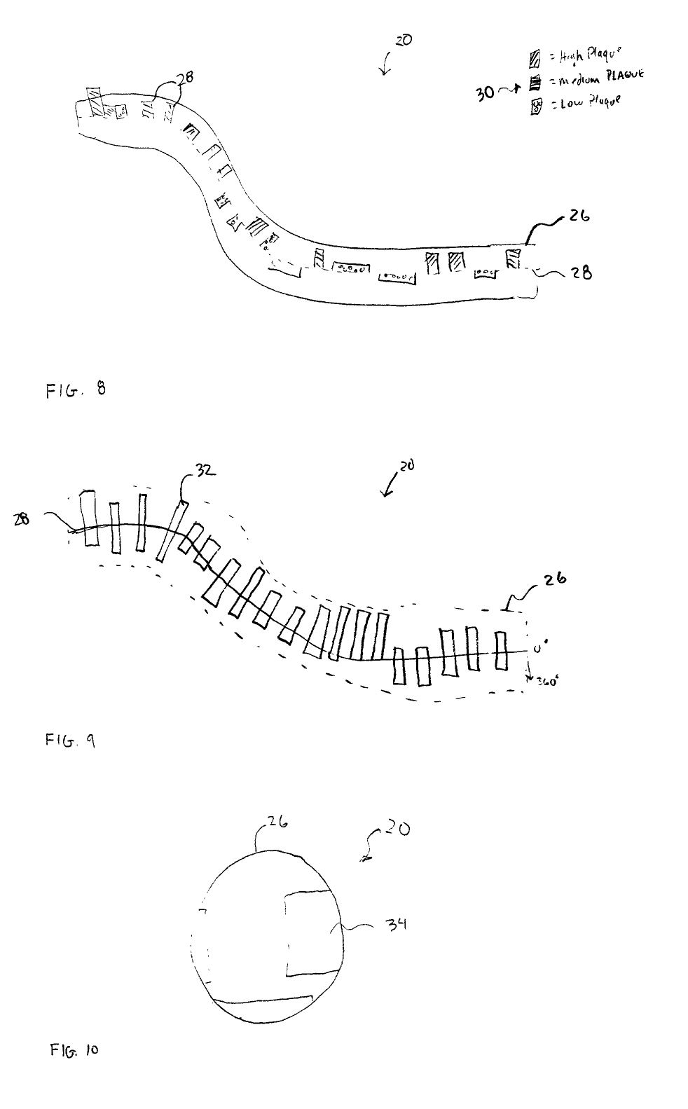

The tortuosity of the body lumen and angulation of the catheter can be determined through analysis of the spacing of the fiducials 24 disposed on the catheter 16. As shown in, FIG. 4, the fiducials 24 will be spaced a known distance X between the adjacent fiducials 24′, 24″, 24′″. During movement through the tortuous body lumen, the fiducials, when viewed in an imaging plane 25 will typically have a reduced separation (FIG. 5). The difference between the actual distance X and the distance viewed X′, X″ in the imaging direction indicates to the operator an average angulation of the catheter in an imaging direction. It should be appreciated that the more fiducials positioned on the catheter, the better angulation and curvature of the catheter can be obtained. Thus if the angulation is needed at finer intervals, the number of fiducials along the catheter can be increased. Note however, that for a single plane angiogram, the fiducials will introduce a 180 degree ambiguity as to whether or not the angulation comes out of or extends into the imaging plane. However, for bi-plane angiograms, it will be possible to track the angulation of the catheter in three dimensions.

The catheter 16 can be positioned at a distal end of the target region prior to the in vivo imaging of the lesion. Once the absolute azimuthal orientation of the catheter is determined a coordinate system can be set. To create a coordinate system along the vessel, the orientation of the catheter can be manually tracked with a fitting program, automatically tracked by a fitting program, or the like. In systems in which that catheter is manually tracked the user can use an input device 18 such as a joystick, mouse, keyboard, or the like (FIG. 1) to move a cursor (viewable on the display 20) to interact with the real-time image of the catheter in the body lumen.

For example, as shown in FIGS. 6 and 7, an operator can mark a plurality of points A, B, C (typically between three and five points) along a coordinate line 28 in the body lumen, typically over the fiducials, (FIG. 6). As can be appreciated, the more points marked the coordinate line will have a greater accuracy. The fiducials can be used to provide a coordinate or dimensional scale to the line thus obtained. The fitting program can fit a straight line or various order curves between the marked points A, B, C. If the coordinate line(s) 28 do not correspond to the shape of the body lumen, the operator can interact with the final fit of the line to make changes, such as adding additional points D, E, F or simply selecting and moving the curve to fit the body lumen (FIG. 7). Fitting the coordinate system to substantially track a longitudinal axis of the body lumen tells the computer where to place the information about the lesion. For systems that incorporate a fitting program, the computer can automatically position a coordinate line that corresponds to the curvature of the body lumen. It should be appreciated that if bi-plane angiography is used this process can be done along two planes and the vessel coordinate system can be traced in a three-dimensional space.

Once the catheter coordinate system is determined, the catheter detectors can obtain information about the lesion and display the information with the anatomic image of the body lumen, most typically along and around the coordinate line 28. Some non-limiting exemplary methods of obtaining information about the lesions is more fully described in U.S. patent application Ser. Nos. 09/754,822, 09/754,074, 09/754,103, all filed Jan. 3, 2001, the complete disclosures of which are incorporated herein by reference.

In one exemplary embodiment, the detector is positioned at a distal end of the target region and the detector is pulled proximally through the target region at a linearly constant speed. The speed of the catheter is tracked by the computer 14 so that the data obtained by the catheter detector(s) can be displayed in the correct anatomical position (both longitudinally and azimuthally) on the anatomic image of the body lumen. In another exemplary embodiment, a position sensitive catheter having a plurality of detectors is positioned at the target region and maintained in a stationary position.

In exemplary embodiments, the information obtained with the intravascular catheter 16 is displayed with the angiographic image. In some embodiments, the information is superimposed directly onto the anatomic image and is positioned in an anatomically correct position on the body lumen. In other embodiments, the information can be displayed as a separate image adjacent the anatomic image of the body lumen.

The present invention provides various methods for displaying the information in a spatially correct presentation over the body lumen such as graphs, histograms, color bars, and the like. For example, as illustrated in FIG. 8 the display 20 can show the body lumen 26 having variable colors 30 to indicate the presence of a temperature difference, a radiolabeled vulnerable plaque or other lesion, biological differences in the body lumen, or the like. In an exemplary embodiment the color range follows a color scale. The different colors of the rainbow can be used to indicate levels of plaque, the type of plaque, or the like. In a specific configuration, blue or violet can be used to indicate no plaque while the other colors of the color map can be used to gradually indicate increasing levels of plaque (or increase in temperature) such that red can indicate the highest levels of vulnerable plaque.

In another embodiment, illustrated in FIG. 9 one side of the coordinate line 28 can carry a (straight or curving) bar histogram 32 representing counts or other indicators of the lesion by the length and/or width of the bars. Typically, a longer bar will indicate more plaque. The second side of the line could carry azimuthal information in the form of another set of bars where the length corresponds to an average angle of the azimuthal position of the plaque with respect to a vertical to the imaging plane (or any other reference). Conversely, the bars could be equal length and the information can be presented by the colors of a color map, as indicated above. This method has the advantage that 0° and 360° degree or −180 and +180 degree transition smoothly in the display, rather than abruptly as they would with bar length representation.

It should be appreciated that a one-dimensional representation of the plaque will typically show an average angle or a “center of mass” angle (e.g., an average angulation) of the azimuthal distribution of the lesion. For two dimensional representations of the plaque, it is possible to provide a second line, color, or bar to show the angular width of the distribution. Thus, for a lesion that extends around the entire circumference of the body lumen, the width of the graph would be maximum. In alternative embodiments, a crosssectional view of the vessel at chosen locations can be used to show the azimuthal distribution of the plaque.

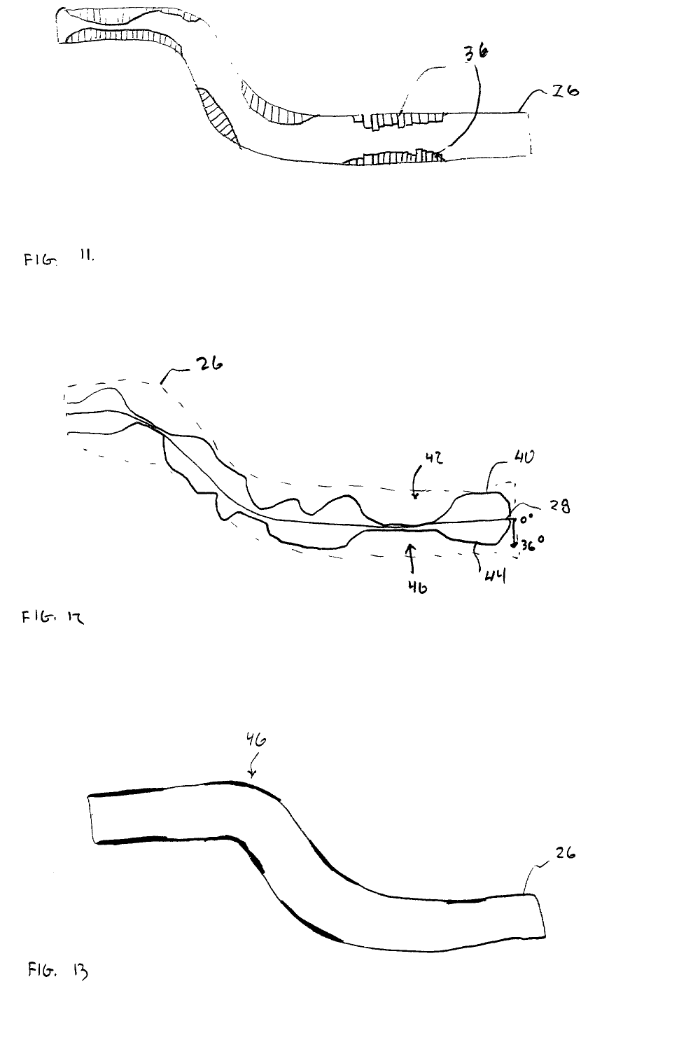

As shown in FIG. 10, the display program can be programmed to allow an operator to display the azimuthal distribution of the lesion or radiation counts at any point of the vessel 26 by displaying the bar lengths or narrowing effects 34 along a circle that represents a cross section of the body lumen.

In other embodiments, the computer 14 will be programmed to allow the user to click on a point or a plurality of points on the anatomic image to display different information. For example, in some embodiments, the combined image will display only the concentration of count activity of the lesion. For example, the image can show a concentration of radiolabels, iodinated contrast media, water, lipid, and the like. If the user is interested in a specific portion of the body lumen, the cursor can move over and click on the specific portion to display additional information, such as azimuthal distribution. Alternatively, the computer can be programmed to be able to toggle between displaying different sets of information, such that the user can toggle between count activity and azimuthal distribution, or the like.

In yet other embodiments illustrate in FIG. 11, the display 20 can illustrate a virtual reality indication 36 of the “narrowing” of the body lumen by providing bars that have a width that indicates a narrowing of the body lumen to indicate where high levels of count or high temperatures are found. Thus the resulting display would show narrowing where there is a high amount of plaque in the body lumen.

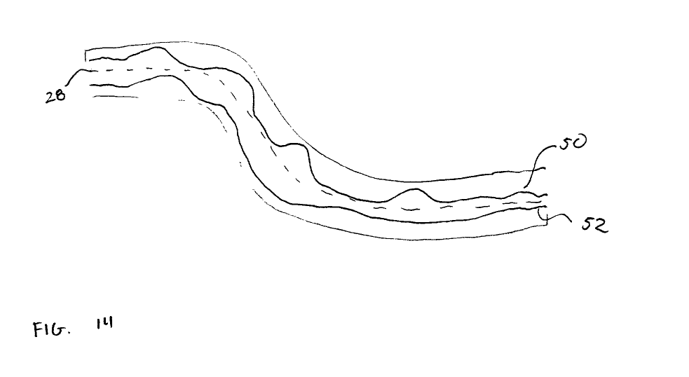

As illustrated in FIG. 12, the coordinate line can carry two sets of information. A first side 38 can include a first graph 40 that indicates the level of plaque, and a second side 42 can include a second line 44 that indicates azimuthal distribution of the plaque (from 0° degrees to 360° degrees).

In yet another embodiment illustrated in FIG. 13, where a black and white display 20 is used, a brightness intensity 46 of the body lumen 26 can be varied to indicate a level of plaque in the body lumen. For example, a brighter line or histogram can indicate high levels of plaque while dim or dark lines can indicate low levels of plaque.

Optionally, as shown in FIG. 14 the displays described above can be enhanced with standard deviation marks 50 (e.g. lines, bars, graphs, brightness variations, or colors) orthogonal to the detector axis 28 or along an outline of the body lumen to highlight the certainty of the data. The standard deviation marks 50 can be shown with vulnerable plaque distribution information (e.g., azimuthal or longitudinal distribution in the form of a bar or graph) or the like. Also, in other embodiments, the computer can be programmed to allow toggling between various information and output displays.

It should be appreciated that in the above methods, a patient's breathing, body movement, heartbeat, and the like may displace the images obtained with the catheter 16 and the anatomic images obtained by the image capture device 12. Consequently, conventional methods, such as rotation, scaling, and displacement of the images can be incorporated into the computer software to register the anatomic image and the information from the catheter. Additionally, computer 14 can be programmed to compensate for different imaging systems, patient breathing, and the patient's heartbeat so that the images obtained from the image capture device 12 and detecting device 16 can be combined. To compensate for different imaging systems at least one of the images can be rescaled so that the images will match both in size and orientation.

To reduce the effect of the patient's heartbeat, the anatomic images of the body lumen are typically captured during the same point in the diastole. For example, imaging with the image capture device 12 and catheter 16 can both be electrocardiographically triggered to a certain point in the diastole. Alternatively, it may be possible to image throughout the patient's electrocardiograph cycle and retrospectively extract data from a certain portion of the cardiac cycle, either by synchronizing with a simultaneously-acquired EKG signal, or by extracting the heart beat interval from periodicities in the acquired data.

To compensate for the displacement of the images due to the patient's breathing the patient may be asked to hold his or her breath. Alternatively, a strain gage belt can be put around the patient's thorax to sense breathing motion, or a flow device can be placed over the patient's nose, and the data segregated into time periods for different parts of the respiratory cycle.

To compensate for rigid body motions (i.e. non distorting movements) radiopaque markers or fiducials (not shown) can be placed on the outside of the patient's body and/or the patient's platform to form a frame of reference. The computer 14 can be programmed to track the position of the markers on both the ghost image and the images acquired by the catheter so as to improve the accuracy of the superimposing of the image. Thus, if needed, the information obtained with the catheter 16 can be adjusted or shifted so that the image obtained with the image capture device 16 match the real time image of the body lumen.

To account for non-rigid motions (e.g. distortions or twisting of the patient's body) the number of fiducials on the patient's body can be increased so that at least two fiducials are along each axis. The computer will track the relative position and orientation of the plurality of fiducials in the real time image obtained by the catheter and will compare the measured distances to the distances of the fiducials in the ghost image. If the relative distances between the markers change, the computer will know that there has been a non-rigid movement in the body. Consequently, the images obtained with the catheter can be modified accordingly (e.g., scaled, enlarged, shrunk, rotated, or the like).

As will be understood by those of skill in the art, the present invention may be embodied in other specific forms without departing from the essential characteristics thereof. For example, the detectors can be used to detect a variety properties and the information can be displayed to the operator in a variety of ways.

Moreover, instead of using a saved image with real time catheter images, a single image taken at a point when opacification has diminished enough to allow the fiducials to be viewed concurrently with the partially opacified vessel.

As another example, a contrast medium can be delivered into the body lumen to capture a location of the markers 24 on the catheter. The catheter position data along with the data acquired with the detectors can be transferred to the computer 14 and the initial contrast angiogram image(s) can be combined by the computer 14 to superimpose the marker location on the coronary images.

Accordingly, the foregoing description is intended to be illustrative, but not limiting, of the scope of the invention which is set forth in the following claims.

What is claimed is:

1. A method of localizing a lesion in a body lumen, the method comprising:
providing an image of the body lumen;
acquiring information about the lesion with a detecting device; and
displaying the information in a spatially correct distribution relative to the image of the body lumen.

2. The method of claim 1 wherein displaying comprises placing the information about the lesion in a substantially anatomically correct position on the image of the body lumen.

3. The method of claim 2 wherein the information is not anatomical.

4. The method of claim 1 wherein acquiring comprises measuring temperature, measuring content of the lesion, or measuring radiation.

5. The method of claim 1 wherein the detecting device comprises an intravascular catheter comprising at least one detector.

6. The method of claim 5 wherein acquiring comprises moving the catheter through the body lumen at a constant speed.

7. The method of claim 5 further comprising determining an absolute azimuthal orientation of the catheter.

8. The method of claim 7 wherein determining comprises rotating the catheter about a longitudinal axis until markers are viewable under radiographic imaging.

9. The method of claim 5 further comprising creating a coordinate system along the body lumen to facilitate placing the information in a spatially correct distribution relative to the lumen.

10. The method of claim 9 wherein creating comprises using a fitting program or a tracing program.

11. The method of claim 5 further comprising localizing the position of the catheter with respect to the body lumen.

12. The method of claim 5 comprising positioning the catheter in a stationary position at a targeted region of the body lumen.

13. The method of claim 1 further comprising delivering a contrast medium into the body lumen, wherein obtaining the image of the body lumen comprises using an MRI system.

14. The method of claim 1 wherein providing comprises obtaining the image of the body lumen with an external imaging system.

15. The method of claim 14 wherein obtaining the image comprises:
releasing a contrast medium into the body lumen during transit of the detecting device to the target site to create an opacified body lumen; and
imaging the opacified body lumen.

16. The method of claim 14 wherein obtaining the image of the body lumen comprises using one of a fluoroscopic imaging system or a CT system.

17. The method of claim 14 wherein obtaining the image of the body lumen comprises using an MRI system.

18. The method of claim 1 or 14 further comprising storing images of the body lumen in a computer memory.

19. The method of claim 18 further comprising combining a plurality of stored images of the body lumen to create the image.

20. The method of claim 1 further comprising marking the lesion in the body lumen with a marker that can be sensed by the detecting device.

21. The method of claim 20 wherein marking comprises binding a radiopharmaceutical to the lesion in the body lumen.

22. The method of claim 21 wherein the lesion is vulnerable plaque.

23. The method of claim 21 wherein acquiring information comprises measuring a count activity of the marked lesion.

24. The method of claim 1 or 23 further comprising adding standard deviation marks on the anatomic image to highlight a certainty level of the information.

25. The method of claim 23 wherein displaying comprises expressing the count activity.

26. The method of claim 1 wherein displaying comprises adding histogram bars onto the image.

27. The method of claim 1 wherein displaying comprises varying a brightness intensity along the lumen to indicate a severity of the lesion.

28. The method of claim 1 wherein displaying comprises providing azimuthal distribution information about the lesion.

29. The method of claim 1 wherein displaying comprises indicating distribution of the marker in the lesion.

30. The method of claim 1 wherein acquiring comprises taking readings only at a specific point in a patient's electrocardiogram cycle.

31. The method of claim 1 further comprising compensating for a patient's heartbeat and breathing during displaying.

32. The method of claim 1 wherein acquiring comprises taking a plurality of readings with the detecting device through a patient's electrocardiogram cycle and selecting data from only a portion of the cardiac cycle for display purposes.

33. The method of claim 1 further comprising tracking a patient's rigid movements.

34. The method of claim 33 wherein tracking comprises placing markers on at least one of a patient's body and a platform beneath the patient's body.

35. A method of characterizing a vulnerable plaque lesion in a body lumen, the method comprising:
externally imaging the body lumen to obtain an anatomic image of the body lumen;
placing detector(s) in the body lumen to distinguish vulnerable plaque from stable lesions; and
displaying the vulnerable plaque information on the anatomic image.

36. The method of claim 35 further comprising generating a coordinate system of the body lumen to orient the count activity information onto the anatomic image of the body lumen.

37. The method of claim 36 wherein generating is carried out with a tracing algorithm.

38. The method of claim 36 wherein generating is carried out with a fitting program.

39. The method of claim 35 wherein placing comprises moving a catheter having the detector(s) through the body lumen.

40. The method of claim 35 further comprising tracking the longitudinal positioning and azimuthal orientation of the detector(s).

41. The method of claim 35 wherein displaying comprises showing at least azimuthal distribution of the vulnerable plaque and a longitudinal position of the vulnerable plaque.

42. The method of claim 35 wherein displaying comprises providing bar histograms representing the amount of plaque by the length of the bars.

43. The method of claim 35 wherein displaying comprises providing a line having a color to indicate the amount of plaque.

44. The method of claim 35 wherein displaying comprises providing standard deviation marks.

45. A method of displaying information about a lesion in a body lumen, the method comprising:
providing an anatomic image of the body lumen with an image capture apparatus;
acquiring azimuthal distribution information about the lesion with a detecting device; and
displaying the azimuthal distribution information with the anatomic image of the body lumen.

46. The method of claim 45 wherein providing comprises externally imaging the body lumen to obtain the anatomic image.

47. The method of claim 46 wherein the detecting device comprises a catheter having detectors.

48. The method of claim 47 wherein the detectors detect one of radiolabel marker counts, temperature levels, MRI parameters, x-ray density, paramagnetic, ferromagnetic or iodinated contrast agents, ultrasound signal, infrared, or optical signature.

49. The method of claim 45 wherein displaying comprises superimposing the azimuthal distribution information over the anatomic image of the body lumen.

50. A system for localizing lesions in a body lumen, the system comprising:
a catheter body comprising a proximal and distal portion;
at least one detector that can obtain information about the lesion;
a plurality of markers positioned on the distal portion of the catheter that allow a user to track the azimuthal orientation of the distal portion of the catheter body; and
a computer that superimposes the information obtained by the detector on an anatomic image of the body lumen.

51. The system of claim 50 further comprising imaging means for obtaining the anatomic image of the body lumen, said imaging means being coupled to the computer.

52. The system of claim 50 wherein the markers comprise a magnetic marker.

53. The system of claim 50 wherein the markers comprises tantalum, gold, or platinum.

54. The system of claim 50 wherein the markers have a shape such that their azimuthal orientation can be obtained in a radiographic image.

55. The system of claim 50 wherein the information comprises at least one of a count activity, azimuthal position of the marked lesion in the body lumen, and longitudinal position of the marked lesion in the body lumen.

56. The system of claim 50 wherein the computer is programmed to position the information in an anatomically correct position on the anatomic image of the body lumen.

57. The system of claim 50 further comprising a display for displaying the information and anatomic image of the body lumen.

58. A system for displaying an anatomic image of a body lumen, the system comprising:
an imaging device that can acquire an anatomic image of the body lumen;
a detecting device to obtain information of a marked lesion in the body lumen; and
a computer configured to store the anatomic image(s) of the body lumen, wherein the computer receives the information from the detecting device and displays the information on the stored anatomic image.

59. The system of claim 58 wherein the detecting device comprises an intravascular catheter comprising at least one radiation detector.

60. The system of claim 59 wherein the catheter comprises a plurality of markers disposed in a spaced relationship on the catheter.

61. The system of claim 58 wherein the anatomic image is an angiographic image.

62. A system for localizing lesions in a body lumen, the system comprising:
imaging means for obtaining an image of the body lumen;
detecting means for characterizing the lesion in the body lumen; and
means for storing the image of the body lumen and superimposing information about the lesion onto the image of the body lumen.
